(19) United States
(12) Patent Application Publication
(10) Pub. No.: US 2007/0210474 A1
(43) Pub. Date: Sep. 13, 2007

(54) Divided Solid Compositions With A High Talc Content, Which Are Intended To Be Incorporated In A Thermoplastic Material

(75) Inventors: Yannick Saint-Gerard, Mougins Le Haut (FR); Frederic Jouffret, Venerque (FR)

Correspondence Address: YOUNG & THOMPSON, 745 SOUTH 23RD STREET, 2ND FLOOR, ARLINGTON, VA, 22202, US

(73) Assignee: TALC DE LUZENAC, LUZENAC (FR)

(21) Appl. No.: 11/578,232

(22) PCT Filed: Apr. 15, 2005
(86) PCT No.: PCT/FR05/00910

§ 371 Date: Oct. 12, 2006

(30) Foreign Application Priority Data
Apr. 20, 2004 (FR) 04.04163

(51) Int. Cl. A61K 9/20; B27N 3/00

(52) U.S. Cl. 264/109; 424/464

(57) ABSTRACT

The invention relates to the preparation of a divided solid composition comprising a talc powder at least a fraction of which consists of grains of talc bonded together in the form of aggregates whose average size is greater than that of said grains of talc. As well as being highly compatible with a large number of thermoplastic resins and having a high talc content, at least of the order of 75%, a divided solid composition obtained according to the invention can be dispersed into fine talc particles in thermoplastic materials during the shaping of these thermoplastic materials (processes with low shear, such as injection moulding, extrusion of sheets/films, etc.) using conventional techniques and equipment. The invention accordingly permits the production of thermoplastic articles loaded with talc without having to use expensive composites (or compounds).

The invention relates to a process for the preparation of a divided solid composition having a high talc content which is to be incorporated and diluted in a thermoplastic material, especially of the olefin type, such as polypropylene and polyethylene. It extends also to compositions so prepared, which are valuable in particular in the manufacture of articles of thermoplastic materials loaded with talc.

In the industry of the manufacture of articles of thermoplastic material(s) it is common practice to incorporate filling agents (for example quartz powder, glass fibres, glass powder, whiskers, talc, calcium carbonate, kaolin, etc.) into thermoplastic raw materials in order to reduce the cost thereof and/or to improve the mechanical properties (especially heat resistance, strength, etc.). This incorporation is mainly carried out by means of a method of mixing by fusion (a) thermoplastic resin(s) and (a) filling agent(s); the method is commonly known by the term “compounding”.

To describe this method schematically, the resin(s) and the filling agent(s) are fed to an extruder, a machine which comprises a heated cover in which there rotate one or more screws whose function is to mix the molten or softened mixture of materials at a high shear rate and force it through a die. A homogeneous mixture emerges from the extruder in the form of rods. The rods are then cooled and cut into granules. The resulting product, in the form of granules, accordingly contains a given amount of filling agent(s) homogeneously distributed in a thermoplastic matrix. This product, which is called a compound or composite, is used by the thermoplastic materials shaping industry, where it serves as starting material for moulding techniques (injection, compression or rotational moulding) and/or extrusion techniques and/or for other methods of manufacturing articles of thermoplastic material(s).

In conventional equipment suitable for these various shaping techniques, the composites are melted and mixed with a very low, or even zero, shear rate, before taking shape during cooling.

With regard to the manufacture of the composites (divided solid mixture of a thermoplastic material and at least one filling agent), because the equipment already in existence in this field is not very suitable for operating with volatile powders, and because talc itself is very volatile when in the form of fine powders, its incorporation into the thermoplastic resins poses numerous problems in practice, in particular:

during weighing and metering, a not inconsiderable amount of powder remains suspended in the air, above the measuring instruments,

during filling of the mixer, phenomena of the talc's being pushed back through the feed hoppers and the formation of a large quantity of dust are observed,

during the mixing by fusion (compounding) in the extruder, some of the talc powder remains suspended in the air inside the machine and does not mix with the thermoplastic resin; another not inconsiderable amount, owing to the very low density of the powder, remains above the layer of resin (which is generally in the form of granules having a much higher density) and is not carried along by the endless screw or screws of the extruder.

For all these reasons, information relating to the content of talc incorporated into the thermoplastic composites, and the articles obtained from these composites, is never very reliable, and the homogeneity of the distribution of the talc particles is very often found to be imperfect.

In order to remedy these disadvantages, which are specific to the handling of volatile powders which are a source of dust, U.S. Pat. No. 4,241,001 proposes bringing the talc powders into the form of granules, which are less pulverulent and easier to transport and then weigh and meter. Subsequently, these concentrated talc powder granules are diluted directly in a thermoplastic material, by mixing by fusion, in order to form a thermoplastic composite loaded with talc. During the mixing by fusion with a thermoplastic resin, by kneading at a high shear rate in the region of the endless screw(s) of the extruder, the talc granules disintegrate and release the talc particles, which then disperse in the molten thermoplastic material. With talc granules having a form and apparent density similar or equivalent to those of the thermoplastic material to be doped (which is generally also in the form of granules), the quality of the composites obtained by mixing by fusion, in an extruder, is improved all the more.

In particular, U.S. Pat. No. 4,241,001 describes a method of compacting powders to form granules, which method comprises adding a wetting agent, especially water, to the powder. The pasty composition so obtained is then converted into granules. It is advisable to add specific agents in order to increase the cohesion of the granules (oily emulsion, molasses-type binder, oily liquid, etc.).

However, the cohesion and stability of the resulting talc granules are such that it is difficult subsequently to redisperse the talc into particles having a fineness similar to that of the grains of the original powder (before granulation), despite the high shear rate of the extruders. One accordingly moves away from the properties originally desired for the composite and for which a particular particle size distribution had been precisely defined for the talc, which particle size distribution has a considerable effect on the mechanical and physical properties of the composite, and especially on those of the final product.

In addition there are also known dispersed solid compositions, called “masterbatches”, which are based on talc and are generally in the form of granules and which consist of a mixture of talc powder (in the preponderant quantity), a thermoplastic resin (for example a polypropylene resin, polyethylene resin, etc.) acting as binder, and a dispersing agent.

Masterbatches are produced in an extruder at a high shear rate, starting from a talc powder or from a compacted talc as described in U.S. Pat. No. 4,241,001, and are generally intended to be mixed by fusion with thermoplastic materials in order to prepare doped thermoplastic composites which will subsequently be shaped to give finished articles. The masterbatches can optionally also be diluted in the thermoplastic materials at the time of shaping thereof by extrusion or moulding techniques (injection, compression or rotational moulding).

The choice of the resin used as binder in such masterbatches is important. The resin must be compatible with the thermoplastic material into which the masterbatch will be incorporated. It often explains the high cost of masterbatches and of the composites obtained using such masterbatches. It governs, on the one hand, the quality of redispersion of the talc particles in the thermoplastic matrix of the composite that is formed; good distribution is an essential component for good quality of the articles ultimately produced. It governs, on the other hand, the optimum talc content in the masterbatches. Nowadays, despite the use of increasingly more powerful kneaders, it is accepted that the optimum content by weight of talc in such masterbatches is relatively low; it scarcely exceeds 70%.

The invention aims to modernize current methods of manufacturing articles of thermoplastic material doped with talc, which methods, as indicated hereinbefore, generally comprise carrying out a succession of three fundamental steps:

1. obtaining a talc powder or compacted talc which is capable of dispersing into a powder in a thermoplastic matrix under the effect of intense shear in an extruder (for compounding),

2. obtaining a composite (or compound), which is often expensive, by mixing by fusion a masterbatch (or an equivalent composition) and a thermoplastic material in an extruder at a high shear rate,

3. obtaining a finished article of doped thermoplastic material by shaping said composite by moulding techniques (injection, compression or rotational moulding) and/or extrusion techniques.

To this end, the main object of the invention is to propose a divided solid composition having a high talc content which is capable of dispersing into a fine powder when it is mixed with a thermoplastic material at a low shear rate (a rate that is much lower than that of an extruder specific for compounding), and in particular during the moulding (injection, compression or rotational moulding) or extrusion (for the purpose of shaping) of the thermoplastic material.

The invention accordingly aims to reform the methods of manufacturing thermoplastic articles loaded with talc in that it allows a thermoplastic material to be doped with talc during its shaping, so that the preparation of a composite (having a given talc content), an intermediate product which hitherto has been essential, is in future superfluous.

Another object of the invention is to propose a divided solid composition having a proportion by weight of talc of at least 75%, the solid part of which benefits from the following characteristics:

suitable cohesion and stability, compatible with normal handling without breaking or crumbling,

form and density of the same order as those of the thermoplastic material to be moulded and doped,

so that it can be mixed directly with thermoplastic materials using conventional equipment suitable for the shaping of thermoplastic materials, while allowing the talc to be redispersed in the thermoplastic matrix with acceptable homogeneity, despite the low shear rate which is characteristic of such conventional shaping equipment.

Another object of the invention is that such a divided solid composition obtained from a talc powder should be usable for and compatible with the doping of a wide variety of thermoplastic materials, especially of the olefin type, such as polypropylene and polyethylene.

Accordingly, the invention relates to a process for the preparation of a divided solid composition which especially is to be incorporated into a thermoplastic material, in particular by a method of mixing by fusion at a low shear rate. Such a composition comprises a talc powder at least a fraction of which consists of grains of talc bonded together in the form of aggregates whose average size is greater than that of said grains of talc.

To this end, in a process according to the invention, the following steps are carried out:

a talc powder in a proportion by weight at least of the order of 75%, based on the total weight of said composition, and a binder, comprising a polyethylene wax, in a proportion by weight at least of the order of 6%, based on the total weight of said composition, are mixed, with stirring; in order to produce said mixture, the talc powder and the polyethylene wax, in solid form, are brought together at a temperature at which said polyethylene wax is in the solid state and, while stirring is maintained, the temperature is raised,

once a homogeneous, pulverulent mixture in which the grains of talc are at least partly encapsulated in the polyethylene wax has been obtained, the mixture is cooled, and

the mixture is granulated by means of a granulating press to form said aggregates.

The term “granulate” is here understood as meaning an operation of extruding the mixture (talc powder and polyethylene wax) through the dies of a granulating press.

With regard to the mixing step, it comprises mixing at elevated temperature, which is easy to carry out, a talc powder and a polyethylene wax initially in solid form, and its aim is to encapsulate the talc particles in a coating of polyethylene wax.

Numerous methods and equipment can be envisaged for this purpose, and these can involve mixers of both the batch and the continuous type.

For example, it is advantageously possible to use mixers equipped with mechanical stirring means and heating means. Mention may be made in this connection of, for example, mixers having a double wall in which a calorific fluid, such as oil, circulates.

During this step of mixing at elevated temperature, when the temperature approaches the melting temperature of the wax, probably starting from the softening temperature thereof, and while the mixture retains a pulverulent appearance, a phenomenon of a more or less sudden collapse of the volume of the mixture is observed.

One explanation for this phenomenon might be the mutual adhesion of the grains of talc, which are covered at least partly with molten wax.

The choice of equipment and the setting of the various operating parameters—especially heating (temperature rise, heating time) and stirring of the mixture (stirring method, speed and time, etc.)—are determined in order to obtain adequate homogeneity of the mixture and avoid the occurrence of a pasty mass.

Advantageously, as soon as a notable reduction in the volume of the mixture is observed, especially a reduction in volume of about half (by simple visual assessment), the step of mixing at elevated temperature is terminated; mixing of the talc and wax is then considered to be sufficiently homogeneous and complete. Of course, it is possible to allow mixing to continue further, but the occurrence of a compressed pasty mass must be avoided.

Advantageously and according to the invention, cooling of the mixture is carried out gradually and continuously, for example at ambient temperature, after removal from the mixer.

Tests carried out by the inventors have shown that, with such a process, and especially thanks to the use of a polyethylene wax, it is possible to obtain concentrated talc aggregates, especially in the form of granules, which advantageously have a content by weight of talc that is at least equal to 75%, while having suitable cohesion, ensuring rigidity and good stability. These mechanical properties manifest themselves in a maintenance of form without breakage or excessive crumbling (without pronounced emission of fines, or dust) during subsequent storage or handling of the aggregates.

Moreover, their dilution in the thermoplastic resins, in particular by mixing by fusion requires only a very low shear rate.

The inventors' works have also demonstrated that the addition of a divided solid composition according to the invention to a thermoplastic material during the shaping thereof—especially by conventional techniques and equipment of injection, compression or rotational moulding or of extrusion—advantageously allowed the steps of doping with talc and of shaping of a finished thermoplastic article to be carried out simultaneously. Although the shear rate is relatively low, its effect, without doubt combined with that of heat, is nevertheless sufficient to disintegrate and disperse the solid part of said composition into fine talc particles in the molten thermoplastic material. It has also been found that the talc particles so dispersed have a fineness that is very similar, or even equal, to that of the grains of the original talc powder (before compaction).

Accordingly, as well as being highly compatible with a large number of thermoplastic resins of the olefin type and having a talc content which can substantially exceed 75%, and therefore being able to constitute a valuable alternative to the use of ordinary talc-based masterbatches, a divided solid composition obtained according to the invention advantageously allows composites loaded with talc to be dispensed with in the manufacture of thermoplastic articles doped with talc.

Advantageously and according to the invention, a polyethylene wax having a molar mass of from 1000 to 50,000 g·mol⁻¹ is used.

With regard to the step of pressing the mixture through the dies of a granulating press, this results in the compaction and granulation of the mixture. Short rods are then obtained, which can advantageously be cut into pieces, at the outlet of the dies, to give aggregates in the form of granules of homogeneous and well defined size.

As granulating presses that are particularly suitable for carrying out the invention there may advantageously be mentioned presses of the KAHL® type (AMANDUS KAHL NACHF., Germany), which have vertical dies and rollers having a vertical axis of rotation.

The invention can also be carried out by means of a granulating press having horizontal dies, such as, for example, presses of the CPM® type (CPM Europe S.A., France).

Advantageously and according to the invention, there is mixed with the talc powder an amount of polyethylene wax suitable for obtaining aggregates in which the proportion by weight of-polyethylene wax is of the order of from 20 to 25%. The content by weight of talc in the aggregates is then of the order of from 75 to 80%, and the binder used here. consists simply of a polyethylene wax according to the invention.

According to another variant for carrying out a process according to the invention, there is produced a mixture of talc and binder in respective amounts suitable for obtaining aggregates in which the proportion by weight of talc is of the order of from 85 to 92%.

Advantageously and according to this variant, there is used in addition to the polyethylene wax at least one surface-active agent selected from: amines, quaternary ammonium salts, quaternary polyammonium salts, carboxylic acids.

Amine is understood as meaning primary, secondary and tertiary amines which can be linear or cyclic, saturated or unsaturated and optionally branched and/or ethoxylated.

The surface-active agent is used in an amount corresponding to a proportion by weight of the order of from 1 to 2% of the weight of the aggregates to be obtained, that is to say of the order of from 1 to 2 wt. % of the mixture to be produced (talc powder, polyethylene wax and surface-active agent).

To this end, before the talc powder and the polyethylene wax are mixed, said talc powder is first heated and mixed with said surface-active agent at a temperature that is at least equal to the melting temperature of said surface-active agent.

Advantageously and according to the invention, said surface-active agent is added in the liquid state to the talc powder.

By covering and functionally modifying the surface of the talc particles, the surface-active agents substantially improve the binding power of the polyethylene waxes according to the invention. Consequently, a very small amount of surface-active agent advantageously allows the amount of polyethylene wax that is used to be reduced substantially and, by contrast, the amount of talc to be increased, with results that are substantially equivalent, or even better, especially in terms of the mechanical stability of the granules. The surface-active agents according to the invention would likewise have an effect in terms of the ability of the talc particles to redisperse in the thermoplastic matrix.

Surprisingly, the inventors have accordingly found that, with approximately 1% (by weight, based on the total mixture) of surface-active agent, it was possible to reduce the amount of wax by the order of 5%.

Accordingly, with this particular variant of the invention, using (a) surface-active agent(s), the inventors have successfully obtained granules of talc powder which can advantageously contain a far higher proportion by weight of talc, which can markedly exceed 85%.

Advantageously and according to the invention, there is used more particularly a surface-active agent selected from: a N,N-bis(hydroxyethyl)amine (for example Noramox® SH2 from CECA, France), a polyether amine, an amine oxide, a betaine, stearin (or stearic acid), a diammonium polydimethylsiloxane (for example Tegopren® 6922, a product from GOLDSCHMIDT, Germany).

Advantageously and according to the invention, a polyethylene wax having a molar mass of the order of 3800 g·mol⁻¹ and a melting point of the order of 100° C. is used. As a polyethylene wax which meets these characteristics there may be mentioned the wax AC617A (HONEYWELL, USA), which has a molar mass of 3796 g·mol⁻¹ and a melting point of 101° C. It is commercially available especially in the form of a white powder.

The invention extends also to products which can be obtained when a process according to the invention, as described above, is carried out. In particular, such products are distinguished in that they are formed from a talc powder converted into aggregates—especially in the form of granules—having a high content by weight of talc, at least of the order of 75%, and which comprise at least of the order of 6 wt. % of a polyethylene wax. The aggregates optionally comprise of the order of from 1 to 2 wt. % of a surface-active agent according to the invention.

The invention relates also to divided solid compositions which comprise products corresponding to this description, and covers more particularly the use thereof in the manufacture of an article of doped thermoplastic material, in particular thermoplastic material doped with talc.

The invention accordingly relates to a divided solid composition which especially is to be incorporated into a thermoplastic material, in particular at the time of the shaping of the thermoplastic material to form a finished article.

Such a composition comprises a talc powder at least a fraction of which consists of grains of talc bonded together in the form of aggregates whose average size is greater than that of said grains of talc. Said aggregates are composed of talc in a proportion by weight at least of the order of 75% and a binder comprising a polyethylene wax. According to the invention, the proportion by weight of polyethylene wax in said aggregates is at least of the order of 6%.

Advantageously and according to the invention, said polyethylene wax has a molar mass of from 1000 to 50,000 g·mol⁻¹.

Advantageously, in a composition according to the invention, the talc aggregates are in the form of granules of homogeneous and well defined size.

According to a particular aspect of the invention, the proportion by weight of polyethylene wax in the aggregates is advantageously of the order of from 20 to 25%.

Advantageously and according to another particularly valuable aspect of the invention, the proportion by weight of talc in the aggregates is of the order of from 85 to 92%. According to this aspect of the invention, the binder comprises, in addition to the polyethylene wax, of the order of from 1 to 2 wt. %, based on the aggregates, of a surface-active agent selected from: amines, quaternary ammonium salts, quaternary polyammonium salts, carboxylic acids.

Advantageously and according to the invention, said surface-active agent is selected from: a N,N-bis(hydroxyethyl)amine (for example Noramox® SH2 from CECA, France), a polyether amine, an amine oxide, a betaine, stearin (or stearic acid), a diammonium polydimethylsiloxane (for example Tegopren® 6922, a product from GOLDSCHMIDT, Germany).

Advantageously and according to the invention, said polyethylene wax has a molar mass of the order of 3800 g·mol⁻¹ and a melting point of the order of 100° C.—especially the wax AC617A (HONEYWELL, USA) of molar mass 3796 g·mol⁻¹ and melting point 101° C.

Advantageously and according to the invention, said aggregates have an apparent density of from 1.2 to 1.7, especially from 1.4 to 1.5.

Finally, the invention extends to a method of manufacturing an article of thermoplastic material doped with talc, in which doping of said thermoplastic material with talc is carried out at the same time as the shaping thereof.

A divided solid composition according to the invention is used for this purpose. It is mixed with the thermoplastic material during the shaping thereof. To this end, the conventional techniques and equipment suitable for moulding, especially injection, compression or rotational moulding, or extrusion are used.

The invention relates also to a process for the preparation of a divided solid composition based on talc, to a divided solid composition based on talc, and also to the use of this composition, characterized in combination by all or some of the features hereinabove or hereinbelow.

Other objects, features and advantages of the invention will become apparent from the detailed description which follows.

The examples described hereinbelow correspond to specific formulations of talc powder aggregates according to the invention, which have been found to have suitable cohesion and stability, compatible with normal handling, substantially without breakage or crumbling.

These aggregates, which comprise a proportion by weight of talc of at least 75%, or even 92%, are in the form of granules composed of small cylindrical pieces of agglomerated talc powder. The apparent density of the granules is generally from 1.2 to 1.7, especially from 1.4 to 1.5 (apparent density measurements carried out according to ISO standard 787/11).

Tests of the dispersion of the talc in a thermoplastic matrix have also been carried out using different compositions of granules according to the invention. The results obtained, which are shown in the tables below, show good redispersion of the talc in the thermoplastic matrices. These results confirm that the granules, which are simple and inexpensive to manufacture compared with masterbatches, can advantageously be diluted homogeneously in molten thermoplastic materials, with a low shear rate. Accordingly, they can advantageously be used during the shaping of such thermoplastic materials in order to dope them with talc at the same time.

Preparation Method

The preparation of a divided solid composition according to the invention takes place in two main steps: mixing and granulation.

The mixing step comprises treating the talc powder with a molten polyethylene wax. Before said wax is incorporated, the talc powder can optionally be treated with a surface-active agent in order to optimize the effect of the wax.

Mixing is preferably carried out by means of a mixer equipped with mechanical stirring means and heating means. In particular, it is possible to use a mixer of the HENSCHEL® type (HENSCHEL INDUSTRIETECHNIK GmbH, Germany), which has a receiver (with a capacity of the order of 500 litres) provided with a covering of hot oil, allowing the mixture to be heated, and a mechanical mixing device, in the present case a two-bladed system. An internal thermal probe allows the temperature of the mixture to be monitored.

It is also possible to use a more rudimentary mixer of the Moritz® type (MORITZ S.A., France) having a capacity of 200 litres.

The mixture is loaded with, for example, 40 kg of talc powder. In particular, talcs having an average particle size distribution of the order of 3.5 μm (for example the talc LUZENAC A20 marketed by LUZENAC NAINTSCH, Austria) and of the order of 1.7 μm (for example the talc Steamic OOS marketed by TALC DE LUZENAC S.A. France) were tested.

The heating means is controlled in order gradually to heat the talc to a temperature that is at least equal to the melting temperature of the polyethylene wax.

The talc, inside the mixer, is stirred continuously.

In the case of the synthesis of talc-powder-based granules comprising an amount of polyethylene wax AC617A (which has a melting point of 101° C.) and without surface-active agent, the control temperature is fixed at about 120° C.

During the rise in temperature of the talc powder, and preferably before it reaches the melting point of the polyethylene wax (101° C. in the case of wax AC617A), the wax, added beforehand., is incorporated into the talc. Of course, the wax can be added while the talc powder is still cold.

The mixture is kneaded efficiently until it is homogeneous, as the temperature of the talc rises.

Good results have also been obtained by adding the polyethylene wax AC617A directly to talc previously heated to 90° C.

There are, therefore, no absolute conditions and parameters which must be complied with in order to obtain a homogeneous mixture according to the invention. Apart from the composition of the mixture, the features described hereinabove or hereinbelow relating to this mixing step in no way constitute conditions which are absolutely necessary. These features relate only to preferred embodiments, which are given by way of non-limiting examples.

In the case of the formulation of granules comprising, in addition to the polyethylene wax AC617A, a surface-active agent, especially of the type Tegopren® 6922, Noramox® SH2 or stearin, such as those already tested by the inventors, heating of the talc is likewise fixed at a temperature of the order of 120° C.

The incorporation of a given amount of a surface-active agent, in particular in the liquid state and especially selected from those mentioned above, is carried out before the incorporation of the polyethylene wax and preferably when the talc is already hot. In fact, it has been observed that treating the talc with said surface-active agents at elevated temperature, at a temperature of the order of from 60 to 80° C., leads to a considerable improvement in the granulation rate (which is multiplied by 10 as compared with treatment in the cold state) and the quality of the granules (which are much less friable).

Once the surface-active agent has been incorporated and the premixture has been kneaded until homogeneous, the polyethylene wax is added.

In these two main ways of carrying out the invention (with and without surface-active agent), once the polyethylene wax has been added, and when the temperature of the mixture reaches a specific level, a rapid reduction in the volume of the mixture is observed. Generally, this reduction in volume occurs well before the temperature of the mixture has reached the melting temperature of said wax. Tests have shown that the temperature marking the change in volume of the mixture can vary as a function of numerous parameters and conditions relating to the mixing operation (content of wax in the composition, speed of the blades of the mixer, distribution of heat within the mixture, etc.).

The mixture is considered to be kneaded sufficiently, that is to say the state of homogeneity of the mixture is considered to have been reached, when, by simple visual assessment, the volume of the mixture has been reduced by about half, relative to its original volume.

Kneading can then be stopped, and the mixture is allowed to cool passively to a temperature that is at least below the softening temperature of the wax, especially outside the mixer, which is still hot.

The granulation step which follows allows the mixture obtained by kneading to be converted into aggregates. It is carried out by means of a granulating press, which can advantageously be of the KAHL® type (AMANDUS KAHL NACHF., Germany) having vertical dies. In particular, the Kahl 33600 press, which is equipped especially with a die of from 3 to 12 mm, can be used.

The flow rates are of the order of from 400 to 500 kg/h. The outlet temperature of the granules is then about 70° C.

The manufacture of a divided solid composition according to the invention can also be carried out by means of a granulating press having horizontal dies, such as, for example, a press of the CPM® type (CPM EUROPE S.A., France).

Talc Distribution Tests

Various divided solid compositions according to the invention have been analysed in respect of the quality of their dispersion in a polypropylene matrix. To this end, pieces of polypropylene doped with talc were produced using a polypropylene of the Novolen 1103K type (BASELL, France) and various divided solid compositions (which had previously been dried following manufacture), in a weight ratio polypropylene/talc granules of the order of 90:10.

Some of the polypropylene/talc mixtures so prepared were injected on a Hercule H 2060-470-200 industrial press (BILLION S.A., France) equipped with a screw having a kneader nozzle, an industrial press conventionally used for shaping composites or thermoplastic materials according to a usual technique of shaping thermoplastic materials by injection.

The injected pieces are here produced under standard plasticizing conditions (temperature, speed, pressure, etc.), preliminary tests having shown that the injection parameters (counter-pressure, temperatures, dwell time, screw speed, decompression before moulding) had no effect on the dispersion of the talc in the resin.

According to this first shaping technique, the resulting pieces are in the form of a plate having a thickness of 2 mm.

Other polypropylene/talc mixtures, instead of being shaped by injection, were extruded on a IDE ME 60/3 single-screw extruder (equipped with a counter-pressure grid having holes of 2.4 mm diameter) and calendered with the aid of a flat die and a SAMAFOR TA 800 calendering bench equipped with a flat 600 mm die. Sheets having a thickness of from 1 to 2 mm are then obtained. The extrusion rates are of the order of 15 kg/h with a speed of rotation of the screw, of the extruder, fixed at 34 rpm, that is to say at a speed (and accordingly a shear rate) far below the speed conventionally used for shaping a thermoplastic material.

Whatever the shaping technique used (extrusion or injection moulding), samples are cut from the formed pieces (plates or sheets) and then compressed to give films having a thickness of 50 μm.

The dispersion of the granules in the polypropylene matrix is evaluated by the number of talc agglomerates visible in these films (undispersed talc), thus demonstrating the quality of redispersion of the talc of the dispersed solid compositions previously obtained.

The agglomerates are counted using a binocular adjusted to the minimum magnification and over a film surface of 4 cm².

By way of reference there is used a composite of Borealis MB 475 U (BOREALIS, France) loaded with 40% talc, which is diluted prior to extrusion (at 190° C.) in a polypropylene resin SABIC 83 MF 10 (SABIC, France) of grade 1.5 in order to obtain a final mixture of 8% talc. No agglomerate of undispersed talc is visible.

For some compositions, in particular for the granules having the highest talc concentration (for which extrusion at 34 rpm did not allow acceptable dispersion), additional tests were carried out in order to try to optimize the dispersion of the talc. The pieces of doped polypropylene starting from such compositions were moulded by extrusion at a speed of rotation of the screw of the extruder fixed at 90 rpm (instead of 34 rpm), an operating speed similar to standard industrial conditions. A substantial improvement in the dispersion is then observed.

The results of the various dispersion tests are summarised in the two tables below.

Table 1 shows the dispersion of the talc in the injection-moulded pieces. Table 2 shows the dispersion of the talc in the pieces shaped by sheet extrusion.

In the tables, the following scale of ratings is used to evaluate the quality of dispersion of the talc:
+++: excellent dispersion of the talc
++: few talc agglomerates
+: large number of agglomerates

−: many agglomerates (redhibitory for use)

TABLE 1

| Talc | | Surface-active | Dispersion |
|---|---|---|---|
| (Steamic | PE wax | agent | (by injection- |
| OOS) | (AC617A) | (Tegopren) | moulding) |
| 75 | 25 | − | +++ |
| 80 | 20 | − | ++ |
| 80 | 19 | 1 | +++ |

TABLE 2

| | PE wax | Surface-active | Dispersion | |
|---|---|---|---|---|
| Talc | AC617A | agent | (by sheet extrusion) | |
| wt. % | wt. % | wt. % | 34 rpm | 90 rpm |
| A20 80 | 20 | | ++ | |
| A20 83.5 | 15 | Noramox SH2 | +++ | |
| | | 1.5 | | |
| A20 85 | 14 | Noramox SH2 | ++ | +++ |
| | | 1 | (1-4 agglomerates) | |
| A20 85 | 14 | stearin | + | |
| | | 1 | (6 agglomerates) | |
| A20 86.5 | 12.5 | Noramox SH2 | + | |
| | | 2 | | |
| A20 90.5 | 7.5 | stearin | − | +++ |
| | | 2 | (>40 agglomerates) | |

1-23. (canceled)

24. A process for the preparation of a divided solid composition comprising a talc powder at least a fraction of which consists of grains of talc bonded to one another in the form of aggregates whose average size is greater than that of said grains of talc,
which process comprises carrying out the following steps:
a talc powder in a proportion by weight at least of the order of 75%, based on the total weight of said composition, and a binder, comprising a polyethylene wax, in a proportion by weight at least of the order of 6%, based on the total weight of said composition, are mixed, with stirring; in order to produce said mixture, the talc powder and the polyethylene wax, in solid form, are brought together at a temperature at which said polyethylene wax is in the solid state and, while stirring is maintained, the temperature is raised,
once a homogeneous, pulverulent mixture in which the grains of talc are at least partly encapsulated in the polyethylene wax has been obtained, the mixture is cooled, and
the mixture is granulated by means of a granulating press to form said aggregates.

25. The process as claimed in claim 24, wherein the step of mixing at elevated temperature is terminated once a notable reduction in the volume of the mixture has been observed.

26. The process as claimed in claim 24, wherein a polyethylene wax having a molar mass of from 1000 to 50,000 g·mol⁻¹ is used.

27. The process as claimed in claim 24, wherein the mixture is cut into pieces at the outlet in order to obtain aggregates in the form of granules of homogeneous and defined size.

28. The process as claimed in claim 24, wherein there is mixed with the talc powder an amount of polyethylene wax suitable for obtaining aggregates in which the proportion by weight of polyethylene wax is of the order of from 20 to 25%.

29. The process as claimed in claim 24, wherein there is produced a mixture of talc and binder in respective amounts suitable for obtaining aggregates in which the proportion by weight of talc is of the order of from 85 to 92%.

30. The process as claimed in claim 29, wherein, before the talc powder and the polyethylene wax are mixed, there is mixed with said preheated talc powder from 1 to 2% of at least one surface-active agent selected from: amines, quaternary ammonium salts, quaternary polyammonium salts, carboxylic acids,
said mixture is produced at a temperature that is at least equal to the melting temperature of said surface-active agent(s).

31. The process as claimed in claim 30, wherein said surface-active agent is used in the liquid state.

32. The process as claimed in claim 30, wherein there is used a surface-active agent selected from: a N,N-bis(hydroxyethyl)amine, a polyether amine, an amine oxide, betaine, stearin, a diammonium polydimethylsiloxane.

33. The process as claimed in claim 24, wherein there is used a polyethylene wax having a molar mass of the order of 3800 g·mol⁻¹ and a melting point of the order of 100° C.

34. The process as claimed in claim 24, wherein, after the mixing step, the mixture is cooled passively.

35. A divided solid composition comprising a talc powder at least a fraction of which consists of grains of talc bonded together in the form of aggregates whose average size is greater than that of said grains of talc, wherein said aggregates are composed of talc in a proportion by weight at least of the order of 75% and of a binder comprising a polyethylene wax, the proportion by weight of polyethylene wax in said aggregates being at least of the order of 6%.

36. The composition as claimed in claim 35, wherein said polyethylene wax has a molar mass of from 1000 to 50,000 g·mol⁻¹.

37. The composition as claimed in claim 35, wherein said talc aggregates are in the form of granules of homogeneous and defined size.

38. The composition as claimed in claim 35, wherein the proportion by weight of polyethylene wax is of the order of from 20 to 25%.

39. The composition as claimed in claim 35, wherein the proportion by weight of talc in the aggregates is of the order of from 85 to 92%.

40. The composition as claimed in claim 39, wherein said binder comprises, based on the weight of the aggregates, of the order of from 1 to 2% of a surface-active agent selected from: amines, quaternary ammonium salts, quaternary polyammonium salts, carboxylic acids.

41. The composition as claimed in claim 40, wherein said surface-active agent is selected from: a N,N-bis(hydroxyethyl)amine, a polyether amine, an amine oxide, betaine, stearin, a diammonium polydimethylsiloxane.

42. The composition as claimed in claim 35, wherein said polyethylene wax has a molar mass of the order of 3800 g·mol⁻¹ and a melting point of the order of 100° C.

43. The composition as claimed in claim 35, wherein said aggregates have an apparent density of from 1.2 to 1.7.

44. The composition as claimed in claim 35, wherein said aggregates have an apparent density of from 1.4 to 1.5.

45. A method of manufacturing an article of thermoplastic material doped with talc, in which said thermoplastic material is doped with talc at the same time as it is shaped; a divided solid composition as claimed in claim 35 is used.

46. The method as claimed in claim 45, wherein there is used a thermoplastic material shaping technique selected from: extrusion, injection moulding compression moulding and rotational moulding.
